(19) United States
(12) Patent Application Publication
(10) Pub. No.: US 2021/0378782 A1
(43) Pub. Date: Dec. 9, 2021

(54) INSTRUMENT HOLDER

(71) Applicants: Tom Thys, Aarschot (BE); André Thys, Aarschot (BE); Andy Thys, Aarschot (BE)

(72) Inventors: Tom Thys, Aarschot (BE); André Thys, Aarschot (BE); Andy Thys, Aarschot (BE)

(21) Appl. No.: 17/407,562

(22) Filed: Aug. 20, 2021

Related U.S. Application Data
(63) Continuation of application No. 16/188,109, filed on Nov. 12, 2018.
Application No. 16/188,109 is a continuation-in-part of application No. PCT/EP2017/069205, filed on Jul. 28, 2017.

(30) Foreign Application Priority Data
Jul. 28, 2016 (EP) 16181765.5

(51) Int. Cl. A61B 90/11; A61B 90/50; A61B 34/30

(52) U.S. Cl. CPC A61B 90/11; A61B 90/50; A61B 1/00149; A61B 2034/305; A61B 2034/301; A61B 34/30

(57) ABSTRACT

A holder for use in combination with an external object is provided, in particular a holder for which the external object moves around a remote center of motion. The external object may be a medical instrument such as a cannula or trocar whereby the remote center of motion is the patient's abdomen. The holder includes two members, generating motion in both a longitudinal and rotational manner. The holder can be used to support another object, in a particular embodiment a camera, convenient for performing endoscopic (laparoscopic) surgery and is equipped with means for generating motion, both in a longitudinal and rotational manner following XYZ coordinates. The holder is particularly useful for working in small areas as it occupies only a small bedside space, without scarifying on functional work area of the instrument mounted in the holder.

### CROSS-REFERENCES TO RELATED APPLICATIONS

This application is a continuation of U.S. patent application Ser. No. 16/188,109 filed Nov. 12, 2018, which is a continuation-in-part under 35 U.S.C. § 111(a) of International Application PCT/EP2017/069205, filed Jul. 28, 2017, which designates the United States and claims the benefit of priority under 35 U.S.C. § 119(a) to European Application Serial No. 16181765.5, filed Jul. 28, 2016.

### TECHNICAL FIELD

The present invention is directed to a holder which has to be combined with an external object whereby the external object moves around a remote center of motion. The external object preferably is a medical instrument such as a cannula or trocar whereby the remote center of motion is the incision in the patient's abdomen. The holder can be used to support another object, in a particular embodiment a camera, convenient for performing endoscopic surgery, including in video-assisted thoracoscopic surgery, laparoscopic surgery and arthroscopic surgery; and is equipped with means for generating motion, both in a longitudinal and rotational manner following XYZ coordinates. The holder of the present invention is particularly useful for working in small areas as it occupies only a small bedside space, without sacrificing on the functional work area of the instrument mounted in the holder.

### BACKGROUND

In standard laparoscopic abdominal surgery, incisions are made into the abdomen, followed by insufflation of the patient's abdomen with gas, and passing through cannulas via the small (approximately ½ inch) incisions to provide entry ports for laparoscopic surgical instruments. The laparoscopic surgical instruments generally include a laparoscope for viewing the surgical field, and working tools such as clamps, graspers, scissors, staplers, and needle holders. The working tools are similar to those used in conventional (open) surgery, except that the working tools are often thin and long with at one end tools working in the surgical field and at the other end handles manipulated by a surgeon. To perform surgical procedures, the surgeon passes instruments through the cannulas and manipulates them inside the abdomen by sliding them in and out through the cannulas, rotating them in the cannulas, and “levering” (pivoting) them around the centers of rotation approximately defined by the incisions in the muscles of the abdominal wall. This point is generally referred to as the Remote Center of Motion (RCM). To maintain accurate positional control of an instrument during surgery, the surgeon may need to manually constrain it to pivot around the RCM coincident with the incision. Manual support of the pivot point is particularly of importance when the surgeon employs laparoscopes or other heavy instruments. Mechanical clamping devices are used to support the instruments in fixed orientations, but these devices do not provide a remote center of rotation for positioning the instruments around the RCM.

Consequently, different RCM positioners have been developed relying on varying approaches to provide a remote center of rotation and assist surgeons in minimal invasive surgery. The present approaches in guiding the instruments around the RCM can be categorized according to the kinematic mechanism used. One of the best-known mechanisms is the one employed in the RCM arm of the daVinci® surgical system (U.S. Pat. No. 7,108,688) and relies on a double-parallelogram to constrain a surgical instrument to move around a fixed center of rotation. The major disadvantage of this double-parallelogram mechanism is that it requires a large amount of working space above the patient.

Other systems employ circular tracking arcs (such as the EndoBot of Rensselaer Polytechnic institute), a fixed isocenter (such as the CLEM of Institut Albert Bonniot), a spherical linkage mechanisms (such as the RAVEN of Univ. of Washington), a highly complex gear train (such as the CoBRASurge of Univ. of Nebraska-Lincoln), a synchronous belt system (such as the MicroHand A of Tianjin University) or a set of parallel linkages pivotably connected to a gripper at fixed distances from the remote center of motion (such as the VESALIUS system described in US2012/0132018). Another robotic system, based on a planar remote center of motion device is described in US2013/0123798, as described in the review article of Kuo & Dai, 2009 “Robotics for Minimally Invasive Surgery: A Historical Review from the Perspective of Kinematics”, in “International Symposium on History of Machines and Mechanisms”, Springer Netherlands, ISBN: 978-1-4020-9484-2; pages 343-351.

It has been an objective of the present invention to provide an instrument holder which does not necessarily needs to have a fixed remote center of motion but has to be combined with an external object (e.g. cannula, trocar, . . . ) whereby the external object moves around a RCM (fixed or not), in particular during video-assisted thoracoscopic surgery, laparoscopic surgery, and arthroscopic surgery; more in particular laparoscopic surgery. In great contrast with US2013/0331644, describing an intelligent autonomous camera control for robotics with medical, military and space applications, requiring multiple members with a multitude of coupling types between said members to enable full spatial positioning of the camera, the instrument holder of the present is much simpler in design with only two base members to allow the external object to move around a RCM. Compared to the system described in US2013/0331644, the present invention differs that the first member is not only configured to allow a longitudinal displacement of its connection with the second member but is equally configured to allow rotation of the first member around its longitudinal axis. In US2013/0331644, a further shoulder member is required instead. This not only has an impact on the space occupied by the instrument holder, but also means that more complex couplings are required between said shoulder member and the further elbow member present in US2013/0331644. No such further intermediate shoulder member is required in the instrument holder of the present invention, significantly reducing the space occupied and by relying on a first member configured to allow both a longitudinal and rotary motion of its connection with the second member along its longitudinal axis, the further design is greatly simplified without loss of functionality.

### SUMMARY

In general, the goal of the present invention is to provide a holder for an object, in a particular embodiment a camera, during laparoscopic surgery or other high precision surgeries, i.e. minimal invasive surgical procedures such as video-assisted thoracoscopic surgery, and arthroscopic surgery, that has to be combined with another object, in a particular embodiment a cannula or trocar that moves around a remote center of motion (the incision in the patient, for example the incision in the patient's abdomen in case of laparoscopic surgery). Expressed differently, the external object is held at an isocenter (its remote center of motion) and connected to the holder wherein the holder is configured to follow the motion of the external object around the isocenter (around the remote center of motion). The RCM may be a fixed or non-fixed RCM.

In a first aspect, the present invention provides a holder, said holder having a first member oriented in line with an axis, for example the X-axis of the XYZ coordinate, with its origin at the remote center of motion. The holder further comprises a second member, which is connected at one end on a pivotable manner (pivotable connector) to the first member and having coupling means at the other end for connecting a certain object (the external object) that is able to move around a remote center of motion, preferably a medical instrument such as a cannula or trocar; Said coupling means may be flexible, wherein said coupling means permit rotation in at least two planes and wherein said coupling means are displaced laterally relative to said X-axis by means of said second member. Furthermore, the first member of the holder is configured to allow a longitudinal displacement of the pivotable connector along said axis. Expressed differently, the holder comprises;—a first member oriented in line with an axis, for example the X-axis of the XYZ coordinate, with its origin at the remote center of motion;—a second member, which is connected at one end on a pivotable manner (pivotable connector) to the first member and having a flexible coupling such as a Bal joint, Hinge joint, Knuckle joint, Pin joint, Cotter joint, Bolted joint, Screw joint, and the like at the other end for connecting a certain object (the external object) that is able to move around a remote center of motion; characterized in that the first member is configured to allow a longitudinal displacement of the pivotable connector along said axis; and to allow said pivotable connector to rotate about said axis.

Per reference to the exemplified embodiments hereinafter, in one embodiment the longitudinal displacement and the circumferential displacement of the pivotable connector along the longitudinal axis of the first member is realized by a fixed position of the pivotable connector at the first member and configuration elements allowing a longitudinal displacement of the first member along its longitudinal axis and allowing rotation of the first member along its longitudinal axis. In another embodiment the longitudinal displacement and the circumferential displacement of the pivotable connector along the longitudinal axis of the first member is realized by a slidable position of the pivotable connector at the first member and configuration elements in the first member including guides for the pivotable connector along its longitudinal axis and configuration elements allowing rotation of the first member along its longitudinal axis. In a particular embodiment the flexible coupling is exchangeable. In this embodiment the second member comprises an adaptor, enabling said flexible coupling to be placed at the free end of the second member.

Where the aforementioned configuration allows the holder to follow the motion of the external object around its remote center of motion, it is a further object of the present invention to use the holder in securing the external object in a desired position. Hence in a further embodiment the holder comprises means to lock the moving members of the holder in-between the manipulation of the external object around its remote center of motion. The moving members including the configuration elements of the first member to allow a longitudinal displacement of the pivotable connector along said X-axis and to allow said pivotable connector to rotate about said X-axis, wherein the configuration elements may allow a linear (3) and/or a concave-curved (4) displacement of the pivotable connection (2) along the longitudinal axis of the first member (100).

The moving members further including the pivotable connection between the first and second member and eventually the flexible coupling between the second member and the external object. In one embodiment the holder comprises means to lock one or more of the moving members of the holder, said moving members being selected from the configuration elements of the first member to allow a longitudinal displacement of the pivotable connector along said X-axis; the configuration elements of the first member to allow rotation of the pivotable connector about said X-axis; the pivotable connection between the first and second member; and the flexible coupling between the second member and the external object. In a particular embodiment the holder comprises means to lock the configuration elements of the first member to allow a longitudinal displacement of the pivotable connector along said X-axis; the configuration elements of the first member to allow rotation of the pivotable connector about said X-axis; and optionally the pivotable connection between the first and second member. In another particular embodiment the holder comprises means to lock the configuration elements of the first member to allow a longitudinal displacement of the pivotable connector along said X-axis; the configuration elements of the first member to allow rotation of the pivotable connector about said X-axis; the pivotable connection between the first and second member; and optionally the flexible coupling between the second member and the external object.

As will be apparent to the skilled artisan, the holder can be used passively, wherein the movement of the holder passively follows the manipulation of the external object around its isocenter. Alternatively, the holder is used actively and controls the movement of the external object around its isocenter. Accordingly, in a further embodiment to the invention, the device comprises means for generating motion, both in a longitudinal and rotational manner following XYZ coordinates, thereby holding a given object, such as a camera.

### BRIEF DESCRIPTION OF THE DRAWINGS

With specific reference now to the figures, it is stressed that the particulars shown are by way of example and for purposes of illustrative discussion of the different embodiments of the present invention only. They are presented in the cause of providing what is believed to be the most useful and readily description of the principles and conceptual aspects of the invention. In this regard no attempt is made to show structural details of the invention in more detail than is necessary for a fundamental understanding of the invention. The description taken with the drawings making apparent to those skilled in the art how the several forms of the invention may be embodied in practice.

FIG. 1 is a schematic overview of one embodiment of an instrument holder according to the invention.

FIG. 2 is a schematic overview of another embodiment of an instrument holder according to the invention.

FIGS. 3A, 3B, and 3C are together a schematic overview of another embodiment of an instrument holder, showing three positions (A, B and C) for an instrument holder according to the invention and caused by the longitudinal displacement of second member (200) along the configuration elements (11, 12) in the first member (100) of the holder.

FIG. 4 is a schematic overview of another embodiment of an instrument holder according to the invention.

FIG. 5 is a schematic overview of an alternative embodiment of an instrument holder according to the invention.

FIG. 6 is a schematic overview of an alternative embodiment of an instrument holder according to the invention.

FIGS. 7A, 7B, and 7C are a schematic overview of an alternative embodiment of an instrument holder according to the invention.

FIGS. 8A, 8B, 8C, and 8D together are a schematic overview of alternative embodiments of an instrument holder according to the invention.

### DETAILED DESCRIPTION

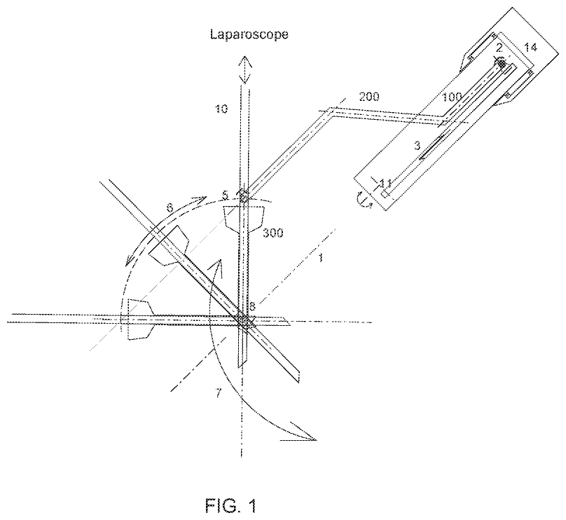

The present invention provides a holder for an (external) object (300), in a particular embodiment a cannula or trocar for minimally invasive surgical procedures, said holder having two members. FIG. 1 is a schematic overview of an embodiment of the holder. In this embodiment the holder in itself does not need to have a remote center of motion, hence when to be used during minimally invasive surgical procedures it has to be combined with another object (300), in a particular embodiment a cannula or trocar that moves around a remote center of motion (the incision in the patient) (8). The instrument holder of the instant application can be used during a minimally invasive surgical procedure selected from video-assisted thoracoscopic surgery, laparoscopic surgery, and arthroscopic surgery; more in particular laparoscopic surgery. In said last embodiment the remote center of motion coincides with the incision in the patient's abdomen. Not being limited to laparoscopic surgery, various tools or instruments like cannulas and trocars having a remote center of motion, like the narrow entry points in laparoscopic surgery or other high precision surgery, can be supported by this invention. In said embodiment wherein the object is a cannula or trocar, various tools, like a laparoscope (10) can be inserted into said cannula or trocar and accordingly held by the holder of the instant application.

Referring to FIG. 1, a schematic overview of an embodiment of an instrument holder according to the invention is shown. The holder includes a first member (100) contained into a fixed body (14) with bearings, a second member (200) and the external object (300) (e.g. trocar, cannula, . . . ) coupled thereto and held spatially constrained at an isocenter (8). In this embodiment the flexible coupling means (5) and the external object (300) are fixed to each other. A laparascope (10) may be inserted into the external object (300).

In the embodiment of FIG. 1, the first member (100) is able to rotate within the fixed body (14) around its longitudinal axis, i.e. an X-axis (1) as herein represented. Furthermore, the pivotable connection (2) between the first (100) and second (200) member follows via a configuration element (linear guide (11)) in the first member (100) a longitudinal displacement along the X-axis (1), allowing a linear (3) displacement along the longitudinal axis of the first member (100). Being spatially constrained at an isocenter (8) the external object should be able to follow a spherical space around said isocenter (8), thus requiring the capability of a rotational movement about a first (indicated by arrow (6)) and a second (indicated by arrow (7)) direction. In the present embodiment of the instrument holder the rotational movement (6) is made possible by the movement caused by the pivotable connection (2) and the flexible coupling (5) together with the longitudinal displacement of the pivotable connection (2) in the linear guide (11), whereas rotational movement (7) is made possible because of the rotational movement of the first member (100) within the fixed body (14) along its longitudinal axis (1). Together these configuration elements enable movement of the flexible coupling according to rotational movement (6) and (7).

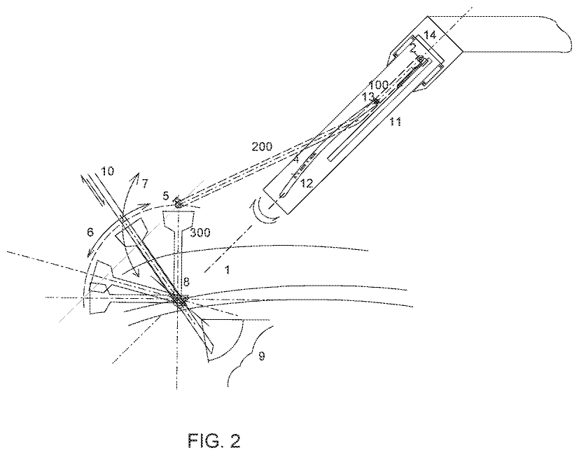

Referring to FIG. 2, a schematic overview is shown of an additional embodiment of an instrument holder according to the invention. The holder includes a first member (100) contained into a fixed body (14) with bearings, a second member (200) and the external object (300) (e.g. trocar, cannula, . . . ) coupled thereto and held spatially constrained at an isocenter (8). In this embodiment the connection (in particular a releasable connection) (5) is a flexible connection (e.g. ball joint). In this embodiment the flexible coupling (5) and the external object (300) are NOT fixed to each other. A laparascope (10) may be inserted into the external object (300), wherein the access area of the external object (300) is the organ (9) of the patient.

In this embodiment, the first member (100) is able to rotate within the fixed body (14) around its longitudinal axis, e.g. an X-axis (1). Furthermore, the pivotable connection (2) between the first (100) and second (200) member follows via a first configuration element (linear guide (11)) in the first member (100) a longitudinal displacement along the X-axis (1), allowing a linear displacement (3) along the longitudinal axis of the first member (100). In addition, in this embodiment, the second member comprises a further guide connector (13) that fits in a second configuration element (12) (a concave-curved guide (12)) in the first member (100), and follows mainly a curved (4) displacement along the longitudinal axis of the first member (100).

Hence, in this embodiment rotational movement (6) is made possible by the movement caused by the pivotable connection (2), the guide connector (13) and the flexible coupling (5), whereas rotational movement (7) is made possible because of the rotational movement of the first member (100) within the fixed body (14) along its longitudinal axis (1), thereby allowing and together with the foregoing configuration elements, movement of the flexible coupling according to rotational movement (6) and (7).

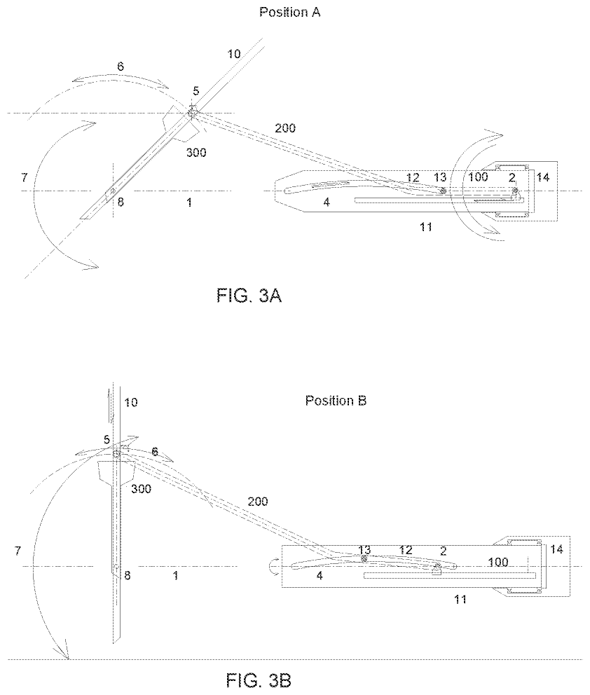

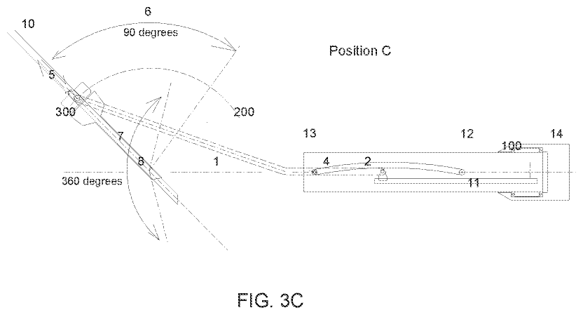

Referring to FIGS. 3A-3C, a schematic overview is provided, showing three positions (A, B and C) for an instrument holder according to the invention and caused by the longitudinal displacement of second member (200) along the configuration elements (11, 12) in the first member (100) of the holder. The position A (FIG. 3A) is the fully retracted position. The position B (FIG. 3B) is an intermediate position. The position C (FIG. 3C) is a fully extended position.

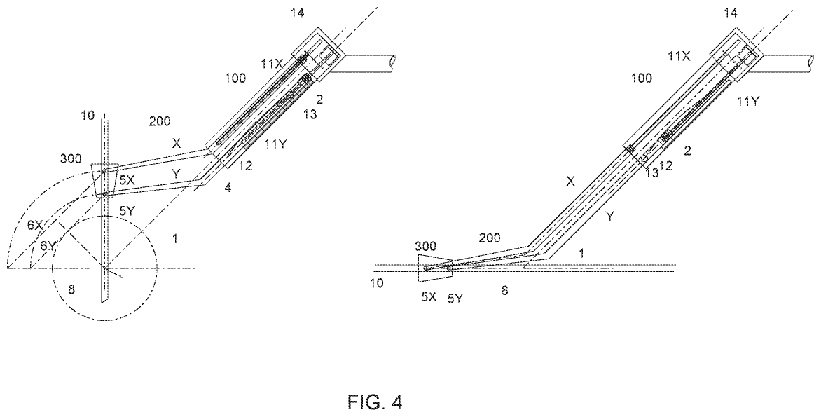

Referring to FIG. 4, a schematic overview is provided of an additional embodiment of an instrument holder according to the invention. The holder includes a first member (100) contained into a fixed body (14) with bearings, a second member (200) and the external object (300) (e.g. trocar, cannula, . . . ) coupled thereto and held spatially constrained at a fixed isocenter (8). In this embodiment, the second member (200) comprises two arms, an upper (X) and lower (Y) arm, each comprising a linear guide (11X), and (11Y) in the first member. The guide of the lower arm (11Y) even comprises a concave-curved guide (12) and a guide connector (13). Furthermore, each arm has its own coupling means (5X and 5Y), said coupling means being fixed together to the external object (300). A laparascope (10) may be inserted into the external object (300).

In this embodiment, the first member (100) is able to rotate within the fixed body (14) around its longitudinal axis, i.e. an axis (1) crossing the isocenter (8) as herein represented. Furthermore, the pivotable connection (2) between the first (100) and second (200) member follows via a configuration element (linear guide (11Y)) in the first member (100) a longitudinal displacement along the X-axis (1). Furthermore, the upper arm X having flexible means near the fixed body (14) enforces the angle of the movement. Being spatially constrained at an isocenter (8) the external object should be able to follow a spherical space (front-, back-, sidewards, . . . ) around said isocenter (8), thus requiring the capability of a rotational movement about a first (indicated by arrow (6X and 6Y)) direction. In the present embodiment of the instrument holder the rotational movement (6X and 6Y) is made possible by the movement caused by the pivotable connection (2) and the coupling means (5X and 5Y) together with the longitudinal displacement of the pivotable connection (2) in the linear guides (11X and 11Y), During this action, the lower arm will follow the rotational movement, whereas the upper arm enables the generation of a fixed isocenter (8). Evidently, speed of both arms contained within the first member varies during movement as it is function of both the rotational movement and the distance between the upper and lower arms.

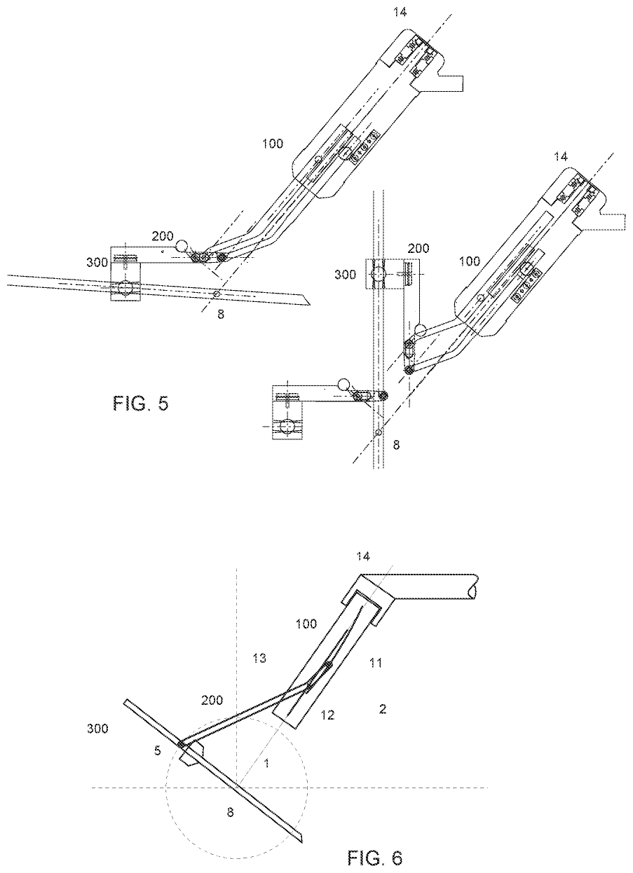

Referring to FIG. 5, a schematic overview is provided of an alternative embodiment of an instrument holder according to the invention. The holder includes a first member (100) contained into a fixed body (14) with bearings, a second member (200) and the external object (300) (e.g. trocar, cannula, . . . ) coupled thereto and held spatially constrained at an isocenter (8). In this embodiment the first member is configured to rotate along its longitudinal axis and comprises two arms (15X and 15Y) arranged at a distance, in parallel to each other and configured to allow a longitudinal displacement along the longitudinal axis of the first member at a fixed ratio with respect to the first member. Said first and second arm are pivotable coupled the second member at respective distances of the isocenter (2X and 2Y) at a ratio equal to the ratio of the translational displacement of the two arms vis-à-vis the first member, and characterized in that the external object is coupled to the second member by means of a flexible coupling (5).

Referring to FIG. 6, a schematic overview is provided of an alternative embodiment of an instrument holder according to the invention. This embodiment is an alternative to the embodiment disclosed in FIGS. 2 and 3. Compared to said embodiment it also provides the longitudinal displacement of the second member (200) along two configuration elements (11, 12) in the first member of the holder. In both embodiments one of the configuration elements consists of a linear guide (11) and the second consists of a concave curved (12) guide along the longitudinal axis of the first member. In both embodiments the second member is connected by means of a pivotable connector (2) to the first member at one end, wherein the first member is configured to allow a longitudinal and circumferential displacement of the pivotable connector (2) along its longitudinal axis through the first configuration element (11) and a further guide connector (13) that fits in the second concave (12) configuration element. It only differs in the orientation of the two configuration elements with respect to one another. Where in FIGS. 2 and 3 the second (12) configuration element is more proximal to the isocenter than the first configuration element (11). It follows from this schematic drawing that the respective orientation of the configuration elements is interchangeable.

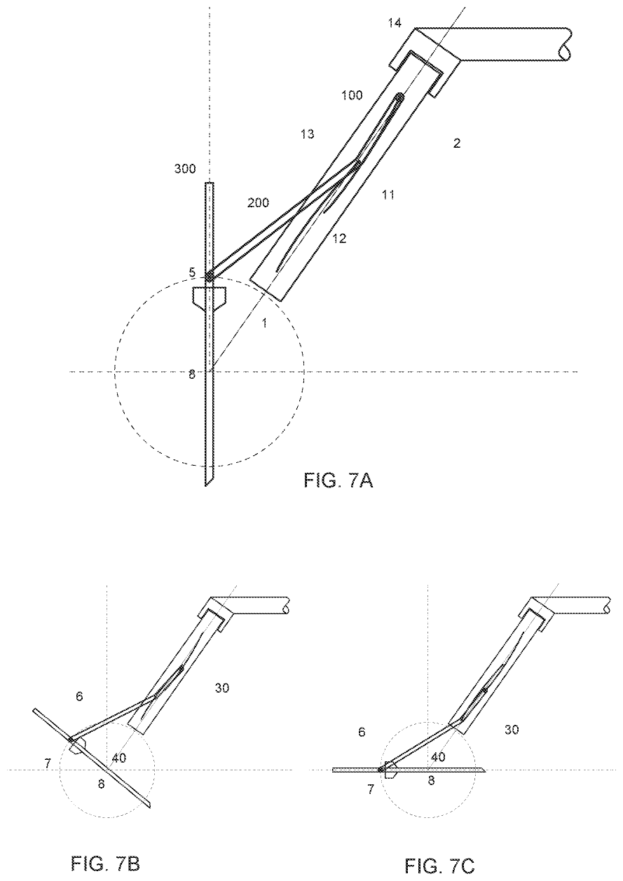

Referring to FIGS. 7A, 7B, and 7C, a schematic overview is provided of an alternative embodiment of an instrument holder according to the invention. This embodiment is an alternative to the embodiment disclosed in FIGS. 2 and 3. Compared to said embodiment it also provides the longitudinal displacement (30) of the second member (200) along two configuration elements (11, 12) in the first member of the holder. In both embodiments the second member (200) is connected by means of a pivotable connector (2) to the first member at one end, wherein the first member is configured to allow a longitudinal (30) and circumferential (40) displacement of the pivotable connector (2) along its longitudinal axis through the first configuration element (11) and a further guide connector (13) that fits in the second concave (12) configuration element. It only differs from the embodiments in FIGS. 2 and 3 in that both configuration elements consist of concave—curved guides ((11) and (12)) that follow mainly a curved longitudinal displacement along the longitudinal axis of the first member (100). It follows from this embodiment that a longitudinal displacement for the pivotable connection (2) of the second member with the first member does not necessarily imply a linear displacement. In FIGS. 7B and 7C two further positions are shown for the longitudinal displacement of the second member (200) along the two configuration elements (11, 12) in the first member of the holder. It follows from these FIGS. 7A-C that this combination of configuration elements together with the pivotable connection (2) allows the coupling (5) to follow rotational movement (6), whereas rotational movement (7) is made possible because of the rotational movement of the first member (100) within the fixed body (14) along its longitudinal axis (1), thereby allowing and together with the foregoing configuration elements, movement of the flexible coupling according to rotational movement (6) and (7). In this embodiment the coupling is preferably a flexible coupling such as a Bal joint, Hinge joint, Knuckle joint, Pin joint, Cotter joint, Bolted joint, Screw joint, and the like

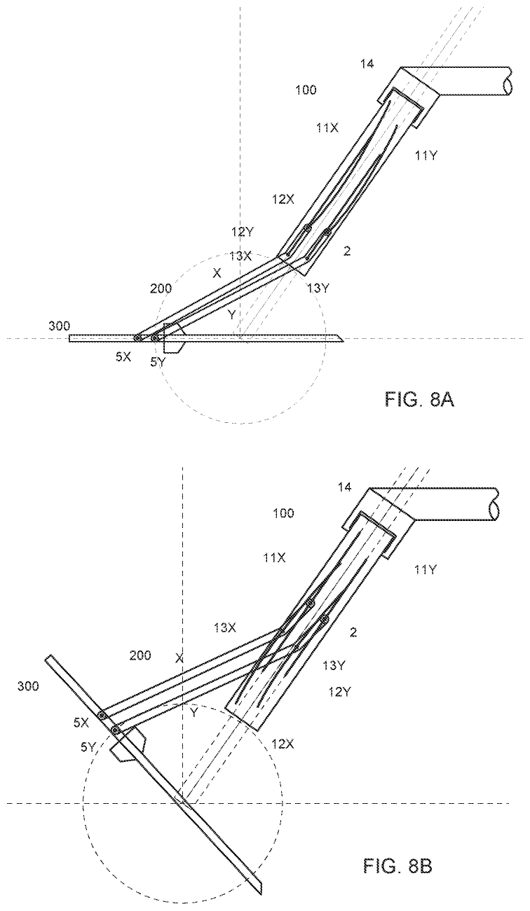

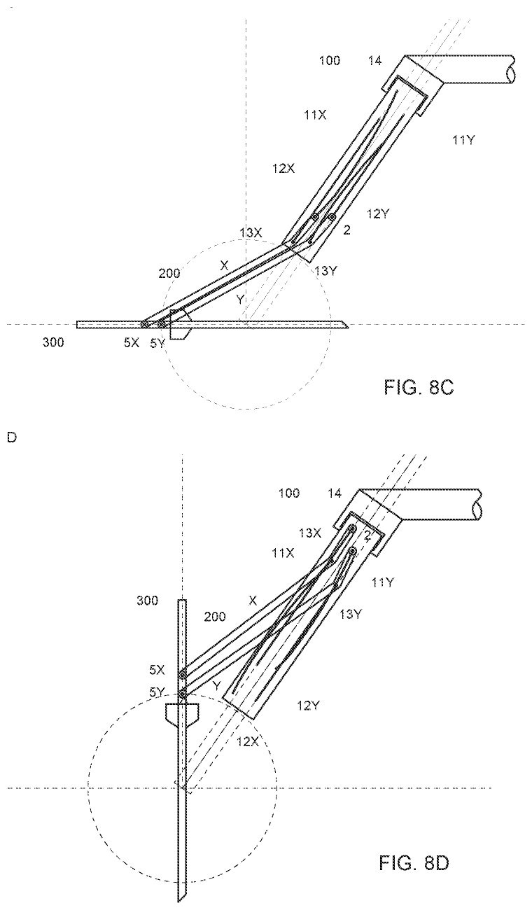

Referring to FIGS. 8A-8D, a schematic overview is provided of alternative embodiments of an instrument holder according to the invention. These embodiments are alternatives to the embodiment disclosed in FIG. 4 wherein the second member (200) comprises two arms (X) and (Y) with configuration elements (11X and 11Y) in the first member allowing a longitudinal displacement of the pivotable connection of such second member (2) along the longitudinal axis of the first member. As for the embodiments with the single arm shown in FIGS. 1-3, 6, and 7A-7C, also for the doubled armed second member, the longitudinal displacement does not necessarily imply a linear displacement but may include concave curved displacements. As for the single armed second member, the configuration elements for the two armed second member include for each arm combinations of linear and concave guides (8A, 8B, 8C and 8D). The concave guides of the arms could run in parallel (8A, 8B) or opposite (8C, 8D) to one another.

As already outlined herein before, the holder of present invention comprises a first member (100) which may be contained within a fixed body (14), and a second member (200) pivotally connected to one another by means of a pivotable connector (2); the second member having a coupling (object coupling) (5) to connect the holder to an (external) object (300) having an isocenter (8), and the first member comprising moving members to allow the object coupling (5) to follow the motion of the external object around the isocenter.

Thereto the first member (100) is oriented roughly in line with the X-axis (1) of the XYZ coordinate with its origin at the remote center of motion (8) of the external object (300). For this initial orientation of the first member along said X-axis, the holder is typically mounted in a tripod or mounting fixture in the proximity of the isocenter of the external object.

The second member (200) is connected at one end on a pivotable manner (pivotable connection) (2) to the first member (100) and has coupling means (5) at the other end for engaging an (external) object (300), a medical instrument like a cannula or trocar in particular. Furthermore, said coupling means may be flexible, thereby permitting rotation in at least two planes and a displacement laterally relative to said X-axis by means of said second member. Furthermore, the first member (100) of the holder is configured to allow a longitudinal displacement of the pivotable connector (2) along the longitudinal axis of the first member and to allow said pivotable connector to rotate about said axis.

In a certain embodiment the longitudinal displacement (30) and the circumferential displacement (40) of the pivotable connector (2) along the longitudinal axis of the first member (100) is realized by a fixed position of the pivotable connector (2) at the first member (100) and configuration elements allowing a longitudinal displacement of the first member (100) along its longitudinal axis and allowing rotation of the first member (100) along its longitudinal axis.

In another embodiment the longitudinal displacement and the circumferential displacement of the pivotable connector (2) along the longitudinal axis of the first member (100) is realized by a slidable position of the pivotable connector (2) at the first member (100) and configuration elements in the first member (100) including guides for the pivotable connector (2) along its longitudinal axis and configuration elements allowing rotation of the first member (100) along its longitudinal axis. Depending on the shape of the guides in the first member, these guides may allow longitudinal displacement in a linear (11), concave-curved (12) manner or combination thereof (infra).

In a particular embodiment, the holder is equipped with means to lock the moving members of the holder in-between the manipulation of the external object around its remote center of motion (8), such as for example with friction couplings for the pivot point (2) between the first (100) and second (200) member, as well as for the configuration elements (e.g. guides 11, 12, 11x, 12x, 11Y, 12Y) of the first member (100) allowing for the longitudinal and rotational displacement of the pivotable connector (2) along the X-axis (1). Once the orientation of the first (100) and second (200) member have been chosen according to the wishes of the person operating the instrument holder, these friction couplings can be locked using a manipulator. In a particular embodiment, the manipulator uses air pressure to lock the friction couplings. In said instances where the external object receives a further instrument (such as for example a laparoscope (10) inserted into a trocar) the former may comprise releasable engaging means to retain said further instrument within the external object in a desired position. In said embodiment, these releasable engaging means of the external object could equally consist of a friction coupling and in a preferred embodiment is being controlled by the same manipulator as the one used to release and engage the friction couplings present on the holder. As such, when releasing the friction components, the manipulator has full control in positioning the further instrument around the center of motion of the external object (300) coupled to the holder of the instant application. To enhance the employability of the holder, in another embodiment the flexible coupling (5) is exchangeable. In this embodiment the second member comprises an adaptor, enabling said flexible coupling to be placed at the free end of the second member.

Furthermore, in a certain embodiment, the pivotable connection (2) between the first (100) and second (200) member follows in its longitudinal displacement along the X-axis (1) a linear displacement, which is the result of a linear configuration element, namely a linear guide (11) in the first member (100) of the holder. In another embodiment, the pivot point (2) connecting the second member to the first member does not merely follow a straight line along the X-axis of the XYZ coordinate with its origin at the remote center of motion of said object (300), when being longitudinally displaced along said axis. In a particular embodiment the longitudinal displacement of said point includes a concave curved (4) displacement, wherein the turning point of the concave is oriented away from said X-axis. In this embodiment, the second member (200) comprises besides the first linearly displaced pivotable connection (2), a further guide connector (13) that fits in a second configuration element (12) (a concave-curved guide (12)) in the first member, and follows mainly a curved (4) displacement along the longitudinal axis of the first member. This embodiment, exemplified in FIGS. 2, 3, and 6 results in a more constrained movement of the holder compared to the embodiment having only a linear displacement and makes it easier for the manipulator to make a more spherical rotation (front-, back-, sidewards) of the object coupling (5) connecting the external object (300) to the second member (200) around the remote center of motion (8) of the external object. In a particular embodiment, the guide connector (13) and the pivotable connection (2) are the same. An even further implementation of such a configuration wherein the pivot point (2) connecting the second member to the first member does not merely follow a straight line along the X-axis of the XYZ coordinate with its origin at the remote center of motion of said object (300), when being longitudinally displaced along said axis, is shown in FIG. 7. In this embodiment both configuration elements (11, 12) are concave curved to demonstrate that it is sufficient for said connection (2) to be displaceable (30) from a distal position to a proximal position with respect to the isocenter directionally along the longitudinal axis of the first member.

From these embodiments it follows that in one aspect of the present invention the holder is equipped with two configuration elements (11, 12) allowing a longitudinal displacement (30) of the pivotable connector (2) along the longitudinal axis of the first member. Said configuration elements could either be linear or concave curved. In the embodiments shown in FIGS. 2, 3, and 6 consisting of the combination of a linear configuration element with a concave curved configuration element; and in the embodiment shown in FIG. 7 consisting of two concave curved configuration elements. For the concave curved configuration elements the turning or inflection point of the concave is oriented away from the longitudinal axis of the first member. In case both configuration elements are curved as in the embodiment shown in FIG. 7, the inflection points are preferably opposite to one another, providing an even more constrained movement of the coupling (5) according to a spherical space around said isocenter (8), thus closer following a rotational movement about a first (indicated by arrow (6)) and a second (indicated by arrow (7)) direction

The second member may be composed of a single arm like in the embodiments shown in FIGS. 1 to 3, 6, and 7 or could be composed of a couple of arms such as presented in FIGS. 4, 5 and 8. Thus in one embodiment according to the invention the second member (200) comprises two arms (X) and (Y). It is at one end pivotably connected through at least one of said arms to the first member, and comprises coupling means (5) such as a ball joint at the other end for holding the external object (300). In a further embodiment each of the arms of such double armed second member (200) have a pivotable connection (2x, 2Y) with the first member at one end and coupling means (5X, 5Y) at the other end for holding the external object. Such double armed embodiments of the second member enhance the stability of the holder at any position of the spherical space around said isocenter (8), In an embodiment wherein the holder comprises a double armed second member, the first member is further characterized in comprising configuration elements (11X, 11Y) to allow a longitudinal displacement of the pivotable connection(s) along the longitudinal direction of the first member for each of the arms (X, Y). As for the single armed embodiments, the first member could independently comprise for each of the arms a second configuration element (12X, 12Y) with a further guide connector (13X, 13Y) present on said arms. Such configuration elements (11X, 11Y, 12X, 12Y) include linear guides, curved guides or any combinations thereof such as a set of linear guides, the combination of linear and curved guides, or a set of curved guides, and can be independently selected therefrom for each of the arms of the double armed second member. Expressed differently, the configuration elements (11X, 11Y, 12X, 12Y)) for each of the arms to allow a longitudinal displacement of the pivotable connection(s) along the longitudinal direction of the first member, are not necessarily the same, see for example the embodiments shown in FIGS. 4 and 8. In a particular embodiment the configuration elements for the double armed second member are the same and run in parallel.

Finally, the holder is made of sustainable materials, which in a certain embodiment are heat stable and therefore suitable for heat sterilisation.

To summarize, the present invention provides a holder, particularly useful as an instrument (camera) holder in minimally invasive surgical procedures and differs from the current RCM manipulators in that the holder in itself does not need to have an RCM. The RCM is present in the object carried by the holder instead (herein also referred to as the external object). In a preferred embodiment this external object is a trocar for laparoscopic or video-assisted thoracoscopic surgery and inserted into the incision of the patient's abdomen or thorax. When inserted this trocar is spatially constraint at said incision point, making this point to behave as a remote center of motion for the trocar. Coupling the object to the holder of the instant application does not further constrain the manipulation of the object. The holder accordingly provides a compact solution to keep a laparoscope or endoscope at a desired orientation, without making concessions on the operational area of manipulation. This large operational area of manipulation is for example apparent in FIG. 2 showing the access area of the external object (300), in said example of a trocar, to the organ (9) in the patient.

What is claimed is:

1. A holder for combining with an external object that moves around a remote center of motion, the holder comprising:
a first member oriented in line with an axis having an origin at the remote center of motion of said object;
a second member connected to the first member at one end through a pivotable connector and a further guide connector and, the second member having coupling means for holding said external object at the other end,
wherein:
the first member is configured to allow a longitudinal and circumferential displacement of the pivotable connector along said axis of the first member; and
the first member is further configured to allow a curved displacement of the further guide connector along said axis of the first member, wherein the turning point of the curve is oriented away from said axis.

2. The holder of claim 1, wherein the longitudinal displacement and circumferential displacement of the pivotable connector along said axis of the first member is realized by a fixed position of the pivotable connector at the first member and configuration elements allowing a longitudinal displacement of the first member along its longitudinal axis and allowing rotation of the first member along its longitudinal axis.

3. The holder of claim 1, wherein the longitudinal displacement and the circumferential displacement of the pivotable connector along said axis of the first member is realized by a slidable position of the pivotable connector at the first member and configuration elements allowing rotation of the first member along its longitudinal axis.

4. The holder according claim 1, wherein the curved displacement of the further guide connector along said axis is realized by a slidable position of the further guide connector at the first member.

5. The holder of claim 1, wherein the longitudinal displacement of the pivotable connector is linear.

6. The holder of claim 2, wherein the longitudinal displacement of the pivotable connector is linear.

7. The holder of claim 3, wherein the longitudinal displacement of the pivotable connector is linear.

8. The holder of claim 3, wherein the first member is equipped with a linear guide for the slidable position of the pivotable connector and a concave-curved guide for the further guide connector.

9. The holder of claim 1, wherein said coupling means are flexible.

10. The holder of claim 9, wherein said coupling means permit rotation in at least two planes.

11. The holder of claim 10, wherein said coupling means are displaced laterally relative to said axis by means of said second member.

12. The holder of claim 10, wherein the rotation in at least one plane of said coupling means is a combination of the longitudinal displacement of the pivotable connector and the curved displacement of the further guide connector.

13. The holder of claim 9, wherein said coupling means are exchangeable.

14. The holder of claim 10, wherein said coupling means are exchangeable.

15. The holder of claim 11, wherein said coupling means are exchangeable.

16. The holder of claim 1, wherein the external object is a medical instrument or medical tool.

17. The holder of claim 1, wherein the external object is a cannula or a trocar.

18. The holder of claim 1, wherein said holder is equipped with means to lock the first and/or the second member.
